(19) United States
(12) Patent Application Publication
(10) Pub. No.: US 2012/0245484 A1
(43) Pub. Date: Sep. 27, 2012

(54) DETERMINING INTRAOCULAR LENS POWER AND POSTOPERATIVE REFRACTION FOR PEDIATRIC PATIENTS

(76) Inventors: Scott K. McClatchey, Jamul, CA (US); Susan M. Whitmer, San Diego, CA (US)

(21) Appl. No.: 13/430,145

(22) Filed: Mar. 26, 2012

Related U.S. Application Data
(60) Provisional application No. 61/467,206, filed on Mar. 24, 2011.

(51) Int. Cl. A61B 3/10; A61F 2/16

(52) U.S. Cl. 600/558; 623/6.11

(57) ABSTRACT

A method for predicting initial postoperative IOL power of a patient undergone IOL surgery. A method for predicting future refractive growth of a pediatric patient's eye.

### CROSS-REFERENCES TO RELATED APPLICATIONS

This application claims priority to U.S. provisional application No. 61/467,206, filed Mar. 24, 2012, which is hereby incorporated by reference in its entirety.

### BACKGROUND

Management of childhood blindness is a priority cited in the “Vision 2020: the right to sight.” Cataract is a major cause of blindness in children throughout the world, particularly in developing countries [1] because of its potential for inhibiting and restricting early visual development. Early surgery now is generally accepted for children with cataract [2], and the placement of an intraocular lens in children undergoing lens aspiration as young as infants is gaining wider acceptance [3,4].

A wise choice of desired postoperative refraction for an individual patient is crucial in the calculation of intraocular lens power. The calculation of intraocular lens power should be as accurate as possible in giving a predictable postoperative refraction—now and in the future. The accuracy of this cataract and ‘refractive surgery’ will permanently enhance the patient's visual life, whereas inaccurate postoperative refractive error may result in lifelong problems.

A number of adult intraocular lens power calculation formulas have been developed and their accuracy reported [5-7], such as the Hoffer Q, Holladay I, Haigis, and SRK/T formulas. An example of a non-pediatric Intraocular Lens (IOL) Calculator is IOL master manufactured by Zeiss (Carl Zeiss Meditec, Inc., Dublin). There is no general consensus as to which formula is the most accurate in children.

Current adult formulas do not take into consideration the continued growth of a child's eye after surgery, which results in a myopic shift, one of the important elements in calculating intraocular lens power in pediatric age group. Myopic shift is a change in eye refraction towards nearsightedness. In normal eyes, axial length increases at a smoothly varying logarithmic curve, so that most of the growth of the axial length is complete by adulthood. In contrast, corneal curvature decreases with age and stabilizes at approximately 1 year of age [11].

Because of the complex functions of the eye, and the numerous factors involved in its refraction, the calculation of the artificial lens' power is somewhat complicated. Axial elongation and changes in corneal curvature are major factors influencing refractive changes in the early childhood. The choice of the power of the appropriate intraocular lens (IOL) for younger children, must take into account of the further growth of the eye, which is impacted by factors including implantation of an intraocular lens into the eye [12].

A pediatric Intra-ocular Lens (IOL) Calculator is computer software used for intraocular lens calculation for young patients. The Pediatric IOL Calculator is based on the Holladay formula, accounting for the logarithmic growth of the eye with the Rate of Refractive Growth (i.e. the RRG formula). This software attempts to predict the refraction of a pseudophakic (a condition in which an aphakic eye has been fitted with an intraocular lens to replace the crystalline lens) child as he/she grows. The model used in this program is based on analysis of the refractive changes in aphakic children (children lacking the natural lenses of the eyes) who underwent surgery before the age of ten with documented refractions for more than 7 years, and it is formulated as a logarithmic model of myopic shift [13, 14]. This program calculates the predicted refraction of a child made pseudophakic, given biometric measurements and intraocular lens parameters. The prediction is shown in graphical form, and allows the surgeon to dynamically view the effects of changing any parameter. It also allows the surgeon to see how closely the actual refractions match those predicted by the program (FIG. 1).

The growth of a child's pseudophakic or aphakic eye results in a myopic shift [12, 24, 5]. The logarithmic model of the rate of refractive growth (RRG) is based on a large, long-term observational case series of aphakic refractions in children [24, 26, 15]. The RRG study found that the mean aphakic refraction follows a simple logarithmic decline from infancy through 20 years of age, with a high correlation (P<0.01, R²=0.97). Because of the asymptotic nature of the logarithmic curve, the model is known to be flawed for the youngest ages (<3 months). In the original model, the value of RRG is defined as the slope of the line of the aphakic refraction at the spectacle plane versus the logarithm of the age. The Pediatric IOL Calculator based on this formula thus does not render calculations for ages younger than 3 months.

In 2010, a new model (RRG2) was developed that “adjusted” the ages by the addition of 0.6 years for measured refraction to account for the growth of the eye in utero [27]. A recalculation of the rate of refractive growth using RRG2 shows that part of the reason for the observed lower RRG values in eyes with surgery prior to 6 months of age is that the values for these eyes are skewed by the asymptotic nature of the RRG model, which means as the age approaches zero, the logarithm of the age approaches negative infinity. If the aphakic refraction truly follows a logarithmic curve down to zero years of age, then the aphakic refraction of a newborn would theoretically approach infinity. However, this estimate is in conflict with reality: an extremely small eye would have extremely high aphakic refraction, but newborns have substantially sized eyes (about 18 mm in axial length) that have been growing for approximately 0.6 years before birth. Because of this known problem with the original logarithmic model (RRG), it is not considered valid before an age of 3 months. Previous studies have subsequently shown that the mean RRG value is lower for those with surgery between 3 and 6 months of age than for those with surgery at or after 6 months of age. The current model of refractive growth (RRG2) addresses the issue of the asymptote at age zero with an age frame shift of 0.6 years to account for the growth of the eye in utero. RRG2 is calculated as the slope of the aphakic refraction at the spectacle plane versus the logarithm of adjusted age. This model was thought to be valid at all ages, even for premature infants. However, on further consideration of the diagrams of aphakic eyes, it was noticed that the smallest eyes could not have ever-increasing aphakic refraction at the spectacle plane. When the vertex distance, which is normally assumed to be 12 mm, equals the focal length of the spectacle lens, the power of the spectacle lens approaches 83 D, and its effective power at the corneal plane grows asymptotically large. Vertex distance is the distance between the back surface of a corrective lens, i.e. glasses (spectacles), and the front of the cornea. Increasing or decreasing the vertex distance changes the optical properties of the system, by moving the focal point forward or backward, effectively changing the power of the lens relative to the eye. In short, using the spectacle plane for the corrective lens introduces a flaw in both the RRG and RRG2 models for the smallest eyes. This results in a large difference between the aphakic refraction measured at the natural lens plane (the plane of the crystalline lens) and the aphakic refraction measured at the spectacle plane. In the example of a hypothetical embryonic eye with a supposed aphakic refraction of +100 D at the spectacle plane as shown in FIG. 2, the vertex distance causes the focus of the spectacle lens to fall between the cornea and the lens, resulting in an effective power of −500 D at the cornea plane. Thus, RRG2 is confounded by an optical artifact due to vertex distance. This explains the observed difference in RRG2 values between pseudophakic children who had cataract surgery earlier than 6 months of age versus at those who had surgery at 6 months of age or older. RRG3 shifts the aphakic refraction to the natural lens plane, from the spectacle plane, to remove the optical artifact inherent in both RRG and RRG2.

### DESCRIPTION OF FIGURES

FIG. 1. An example of prediction curves generated by the Pediatric IOL Calculator (published in 1997).

FIG. 2. The Optical Artifact Caused by Vertex Distance.

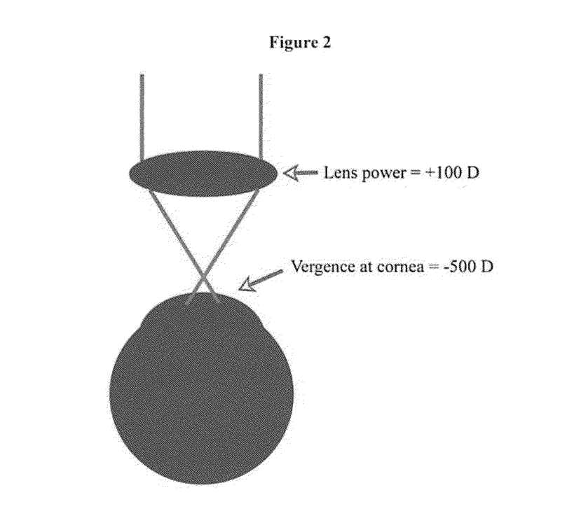

FIG. 3. RRG3 vs. Percent of Eyes. Distribution of the rate of refractive growth (RRG3) values for individual eyes.

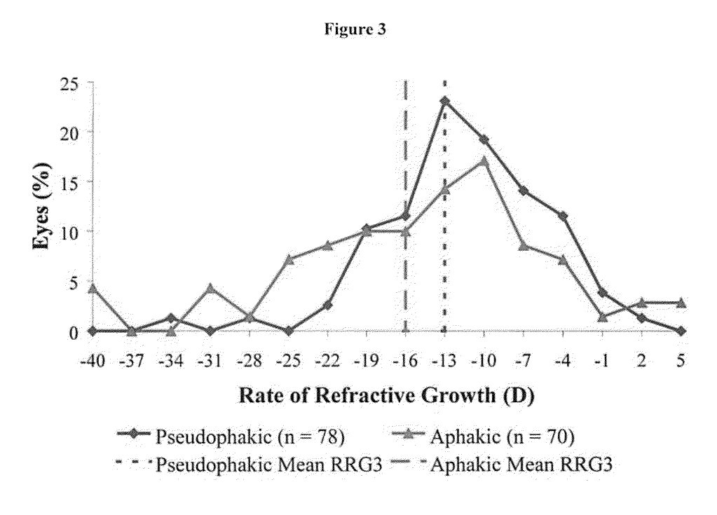

### DESCRIPTION OF THE INVENTION

Precise IOL power calculation is essential for optimal benefit of implant surgery. When making a selection of the proper IOL, some surgeons choose initial hyperopia to reduce the child's future myopia. Other surgeons choose to make a child's eye initially emmetropic (no refractive error) or slightly myopic, in order to reduce the difficulty of amblyopia management.

### Determining Rate of Refractive Growth (RRG)

In order to eliminate the optical artifact of vertex distance from the current model (RRG2), this invention elected to mathematically shift the position of measured refraction for the eyes by developing a new model of refractive growth based on the aphakic refraction calculated at the natural lens plane instead of at the spectacle plane. This model was designed to eliminate the optical artifact due to vertex distance, and provides a better prediction of future postoperative refractions, even in the youngest infants. Instead of spectacle power, the intraocular lens (IOL) power for emmetropia was used to calculate RRG3. Just as a spectacle lens corrects a refractive error at the spectacle plane, an IOL for emmetropia (a state of proper correlation between the refractive system of the eye and the axial length of the eyeball, rays of light entering the eye parallel to the optic axis being brought to focus exactly on the retina) corrects the refractive error at the natural lens plane. Since this plane maintains approximately the same relative position with the eye as the eye grows, the IOL power for emmetropia is not subject to the optical artifact of vertex distance that affects the RRG and RRG2 models. The rate of refractive growth of this invention is determined as

$$RRG 3 = \frac{{AdjAR}_{2} - {AdjAR}_{1 }}{log ( {AdjAge}_{2} ) - log ( {AdjAge}_{1} )}$$

Wherein “RRG3” is the rate of refractive growth, “AdjAR” is the adjusted aphakic refraction or IOL power for emmetropia, and “AdjAge” is the patient's age at the measured refraction plus 0.6 years to account for the time the eye is growing before birth. The subscripts “1” and “2” refer to the initial and final measurements, respectively.

In an alternative embodiment, the rate of refractive growth can be calculated at the corneal plane, which gives an equally valid result, though the measured values for this rate of refractive growth would be different from the measured values of RRG3 [24]. Accordingly, AdjAR₁ and AdjAR₂ will change.

### Determining IOL Power for Emmetropia at Natural Lens Plane

The current IOL formulas are designed for use in adults, and are less accurate when applied to children [24, 27, 28]. Several studies have been conducted to test their validity when applied to children. The mean absolute errors in many of these studies ranged from 1.06 D to 1.4 D in children, which was higher than the mean absolute error of 0.5 D to 0.7 D found in adults. More recent studies have found mean absolute prediction errors of 0.76 to 1.18 when applying the adult IOL formulas to pediatric patients, still with only 43% of eyes with less than 0.5 D of error.

In order to formulate a model that is valid for even the smallest eyes of premature infants, this invention provides a new IOL power calculation formula (W) to determine IOL power.

IOL power (W)=Vergence_{back of IOL}−Vergence_{front of IOL}

The new IOL power (W) formula system assumes that the eye grows proportionately, with the radius of curvature of the anterior cornea (Rak), radius of curvature of the posterior cornea (Rpk), and thickness of the cornea (K_t) determined as a function of the axial length (AL). An upper limit of 8.9 mm is placed on the radius of curvature of the anterior cornea, which is commonly known in the art. The cornea stops growing early in life, and it can naturally grow to the size of approximately 8.9 mm. Highly myopic eyes are generally near-sighted because of axial growth alone. The anterior chamber depth (ACD) is also calculated from the axial length, and the A-constant (A_const) of the specific IOL is provided based on the type and brand of IOL to be implanted. From these parameters, and the known indices of refraction of the cornea (n_k), vitreous (n_vit), and aqueous (n_aq), and the vertex distance from the cornea to the spectacle plane (vertex), the IOL power (IOL_power) at the natural plane for the desired spectacle refraction (PPspecRxSP) can be calculated as follows:
Step 1: Take measurements of biometric parameters of the patient's eye, including but not limited to Axial length (AL), Aphakic Refraction (AR), cornea power (K).
Step 2: Define the constants, indices of refraction, and vertex distance (these constants are known in the art).

n_k=1.3771 (1)

n_vit=1.336 (2)

n_aq=1.3374 (3)

vertex=0.012 (4)

Step 3: Calculate the radii of curvature of the anterior and posterior cornea and the thickness of the cornea based on the known/given parameters and AL. These parameters are calculated as a function of axial length and follow a generic formula:

(parameter)*(AL/0.0235) (5)
Millimeters are converted to meters:

parameter/1000 (6)

Depending what parameter (i.e. radius of curvature of the anterior cornea (Rak), radius of curvature of the posterior cornea (Rpk), or the thickness of the cornea (K_t)) is being calculated, the generic formula (5) can be modified as follows:
Radius of curvature of the anterior cornea (m) with an upper limit:

$$\begin{matrix}Rak = \frac{( 7.8 ) ( \frac{AL}{0.0235} )}{1000} & ( 7 ) \\ If Rak > 8.9 , then Rak = \frac{8.9}{1000} & ( 8 )\end{matrix}$$

Radius of curvature of the posterior cornea (m):

$$Rpk = \frac{( 6.5 ) ( \frac{Rak}{0.0078} )}{1000} \qquad ( 9 )$$

Central cornea thickness (m):

$$K\_t = \frac{( 0.55 ) ( \frac{Rak}{0.0078} )}{1000} \qquad ( 10 )$$

Anterior chamber depth calculated from the A-constant and the axial length (m):

$$ACD = \frac{[ ( 0.58357 ) ( A\_const ) - 63.896 ] ( \frac{AL}{0.0235} )}{1000} \qquad ( 11 )$$

Step 4: Calculate the power of the cornea from its radius of curvature:
   Power of the anterior cornea (D):

$$Pak = ( n\_k - 1 ) ( \frac{1}{Rak} ) \qquad ( 12 )$$

Power of the posterior cornea (D):

$$Ppk = ( \frac{n\_aq}{n\_k} - 1 ) ( \frac{1}{Rpk} ) \qquad ( 13 )$$

Step 5: Calculate the IOL power (W) based on the vergence at the different planes and the above parameters:
   Vergence at the spectacle plane (Vspectacleplane) is the desired spectacle refraction (PPspecRxSP):

Vspectacleplane=PPspecRxSP (14)
Typically, the desired spectacle refraction (PPspecRxSP) is selected based on the surgeon's preference and discussions with the patient. Some surgeons choose initial hyperopia to reduce the child's future myopia. Other surgeons choose to make a child's eye initially emmetropic (no refractive error) or slightly myopic, in order to reduce the difficulty of amblyopia management.
Vergence at the front of the cornea (VfrontK) is calculated based on the desired spectacle refraction and vertex distance. If the desired spectacle refraction is zero, the vergence at the front of the cornea is also zero:

If PPspeckRxSP=0, then VfrontK=0 (15)

$$If PPspecRxSP \neq 0 , then VfrontK = \frac{1}{\frac{1}{PPspecRxSP} - vertex} \qquad ( 16 )$$

Vergence at the back of the anterior cornea (VbackantK) is the vergence at the front of the cornea added to the power of the anterior cornea (Pak):

VbackantK=VfrontK+Pak (17)
Vergence at the front of the posterior cornea (VfrontpostK) is the vergence at the back of the anterior cornea adjusted by the change in indices of refraction and the corneal thickness:

$$VfrontpostK = \frac{n\_k}{\frac{n\_k}{VbackantK} - K\_t} \qquad ( 18 )$$

Vergence at the back of the posterior cornea (VbackpostK) is the vergence at the front of the cornea added to the power of the posterior cornea (Ppk):

VbackpostK=VfrontpostK+Ppk (19)
Vergence at the front of the IOL (VfrontIOL) is the vergence at the back of the posterior cornea adjusted by the change in indices of refraction and the anterior chamber depth:

$$VfrontIOL = \frac{n\_aq}{\frac{n\_aq}{VbackpostK} - ACD} \qquad ( 20 )$$

Vergence at the back of the IOL (VbackIOL) is calculated based on the change in index of refraction, the axial length, the anterior chamber depth, and the thickness of the cornea:

$$VbackIOL = \frac{n\_vit}{AL - ( ACD + K\_t )} \qquad ( 21 )$$

The IOL power (IOL_power) is the difference in the vergences at the back of and the front of the IOL:

IOL_{—} power=VbackIOL−VfrontIOL (22)

### Method for Predicting Future Refraction of a Child Undergo IOL Implantation

In an embodiment of the inventive method for predicting future refraction of a given patient undergoing IOL implantation, the surgeon first measures the following biometric parameters of the patient's eye before surgery. The biometric parameters measured included but not limited to axial length (AL), cornea power (K) or aphakic refraction (AR). Corneal power is typically measured by keratometry. Keratometry should be done for both eyes. It is advisable to repeat measurement if the
a. Average keratometry (K) in either eye is less than 40 D or greater than 47 D.
b. Difference in K between the two eyes is greater than 1 D.
   Alternatively, corneal topography may also be utilized. In young children the measurement of the axial length is best done with A-scan ultrasonography. It can be performed by an immersion technique or a contact technique.

After obtaining these measurements, the surgeon will consider the type and brand of IOL to be implanted for the patient and the desired postoperative spectacle refraction. IDLs made of different materials (PMMA, Acrylic, Silicone etc.) and with different design considerations (Allergen S140, Alcon SA60, AcrySof MA60 etc.) are currently available on the market. Once the suitable IOL lens is picked, the surgeon knows the A-constant value of the IOL and chooses an initial postoperative refraction for the patient. This is the postoperative refraction desired immediately after the surgery. This decision may be based on the surgeon's past experience and the discussion with the patient, among other considerations. Some surgeons will choose to aim for moderate hyperopia while others will choose emmetropia (no initial refractive error) or a small amount of myopia.

In one embodiment, all of the parameters except IOL power (IOL_power) are measured and known before the surgery. In an alternative embodiment, the radius of curvature of the anterior cornea (Rak), radius of curvature of the posterior cornea (Rpk), and thickness of the cornea (K_t) are determined as a function of the axial length (AL) assuming their growth is proportionate to the growth of AL.

The surgeon enters the measured parameters and A-constant, and uses the W system of formulas to calculate IOL power for the chosen initial refraction. In addition, the invention uses the known mean value of pseudophakic RRG3 to predict future refractions of this child at a future age. The mean value of pseudophakic RRG3 of −13±6 is determined based on measured RRG3 from a retrospective case study. The mean value of pseudophakic RRG3 can be refined and modified using additional data from other retrospective studies of pediatric patients. RRG3 value was calculated as the difference in the adjusted aphakic refractions divided by the difference in the logarithms of the adjusted ages. Typically the growth of the eye will result in a logarithmic decline in refraction, with a rapid shift to myopia in the youngest years that tapers off with age. The inventive method calculates the upper and lower standard deviation curves of predicted future refractions, based on the measured standard deviations from the observational

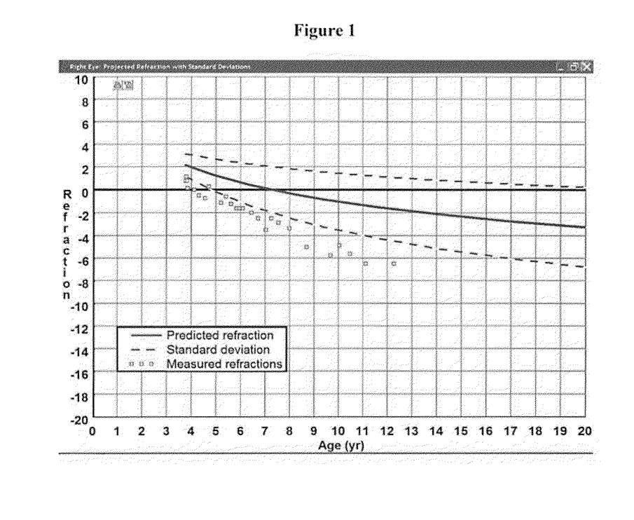

RRG3 study. FIG. 1 shows a sample graph from the Pediatric IOL Calculator that used to make these calculations, which uses the original RRG model and the Holladay formula for IOL power calculation.

An embodiment of the current invention is a similar calculator capable of predicting future refractive growth of a pediatric patient by constructing future refraction curves using the RRG3 model and the newly developed W formula.

$$RRG 3 = \frac{{AdjAR}_{2} - {AdjAR}_{1}}{log ( {AdjAge}_{2} ) - log ( {AdjAge}_{1} )}$$

Step 1: selecting a desired initial postoperative refraction, which is AdjAR₁, the IOL power for emmetropia at the age of surgery;
Step2: calculating AdjAgeᵢ, which is the age at surgery +0.6 years;
Step 3: constructing the predicted pseudophakic refraction curves, wherein the adjusted aphakic refraction (IOL power for emmetropia) is calculated from the age at surgery through at least age 20 years, with an approximate increment of 0.1 years between steps.
   i. At each point (each step in the ages in (c)), the AdjAge₂ is the age at that point +0.6 years.
   ii. At each point (each step in the ages in (c)), AdjAR₂ is calculated via a transform of the formula for RRG3: AdjAR₂=RRG3*(log(AdjAge₂)−log(AdjAge₁))+AdjAR₁
   In order to calculate the refraction of the pseudophakic eye at future ages, the invention calculates the AdjAR₂, at that age, given the values for IOL power, A-constant, and the same assumptions of proportional growth that used in the RRG3 study.
Step 4: The resulting series of data points (consisting of predicted pseudophakic refractions and ages) is plotted to obtain the predicted curves of pseudophakic refraction vs. age.
Step 5: The surgeon inspects the curves of predicted pseudophakic refraction vs. age for the pediatric patient, and elects whether to modify the goal postoperative refraction (and thus change the IOL power) to give a better outcome for the child.

### EXAMPLE 1

### Retrospective Study Validating RRG3 Formula

Data collected in previous studies of pseudophakic and aphakic children are used to validate the new RRG3 formula. The entry criteria were as follows: (1) children 10 years old or younger at the time of cataract surgery, and (2) follow-up time between measured refractions of at least 3.6 years and at least the age at first refraction plus 0.6 years.

For the primary outcome measure, data were extracted, including: side of the surgery (right or left eye), age at surgery, age at initial refraction following surgery, initial refraction, age at final refraction following surgery, and final refraction; for pseudophakic eyes, The IOL power and A-constant were also extracted. All refractions were measured or calculated to be at the spectacle plane. All contact lens refractions were converted to the refraction at the spectacle plane, assuming a vertex distance of 12 mm.

For secondary outcome analysis, information extracted included (when available): age at surgery, best corrected visual acuity (BCVA), sex, uni-versus bi-laterality of the surgery, presence of glaucoma, presence of IOL, and calculated initial adjusted aphakic refraction. For bilateral cases, only data from the right eye were used.

For each measured refraction, the adjusted aphakic refraction was calculated, which is defined as the power of an IOL with an A-constant of 118.4 that would be required to make the eye emmetropic, using the W formula. From the adjusted aphakic refraction, the RRG3 value was calculated as the difference in the adjusted aphakic refractions divided by the difference in the logarithms of the adjusted ages.

For the primary outcome analysis, unpaired two-tailed t-tests were performed assuming equal variances to compare the mean values of RRG, RRG2, and RRG3 for the following groups: (1) pseudophakic patients less than 6 months of age at surgery versus pseudophakic patients 6 months of age or older at surgery, and (2) aphakic patients less than 6 months of age at surgery versus aphakic patients 6 months of age or older at surgery. Unpaired two-tailed t-tests assuming equal variances were then performed for all pseudophakic patients versus all aphakic patients. For all t-tests, a P value 0.05 was considered statistically significant.

Backward stepwise multiple regression analysis was used to analyze whether RRG3 was affected by the following secondary factors: age at surgery, BCVA, sex, uni-versus bi-laterality of the surgery, presence of glaucoma, presence of IOL, and calculated initial adjusted aphakic refraction.

Seventy-eight pseudophakic and 70 aphakic eyes met the entry criteria. The age at surgery ranged from 0.25 to 9 years, with a mean follow-up time of 9.5 years. Characteristics of the study eyes are shown in Table 1. The demographics of the two groups were similar.

TABLE 1
Characteristics of study eyes.

| | | | Mean Time | |
|---|---|---|---|---|
| | | | Between | |
| | Age at | Age at | Measured | Mean logMAR |
| | Surgery | Surgery | Refractions | BCVA (Snellen |
| | (years) | <6 Months | (years) | notation) |
| Pseudophakic Eyes | 0.25-6.1 | 24% | 7.9 | 20/58 |
| Aphakic Eyes | 0.25-9.0 | 31% | 11.3 | 20/74 |

*BCVA is the best-corrected visual acuity at the spectacle plane.

The mean RRG3 value was not significantly different for pseudophakes who had surgery before 6 months of age versus at 6 months of age or older (−11±4 D versus −14±7 D, P=0.12). The mean RRG3 value was also not significantly different for aphakes who had surgery before 6 months of age versus at 6 months of age or older (−15±9 D versus −17±10 D, P=0.61).

Because the mean values for RRG3 in the group of less than 6 months of age at surgery and the group of 6 months or older for both pseudophakes and aphakes were not significantly different, all ages were grouped together for further analysis. The mean RRG3 value for pseudophakic eyes of all ages was −13±6 D versus −16±10 D for aphakic eyes of all ages (P=0.01) (FIG. 1, Table 2).

TABLE 2
Comparison of RRG3 in pseudophakes and aphakes, using the t-test.

| | Age at | | | | | |
|---|---|---|---|---|---|---|
| | Surgery | Number | | | Mean RRG3 (D) at | |
| | (years) | of Eyes | Mean RRG3 (D) | | All Ages | |
| Pseudophakic Eyes | <6 months | 19 | −11 ± 4 | P = 0.12 | −13 ± 6 | P < 0.01 |
| | ≧6 months | 59 | −14 ± 7 | | | |
| Aphakic Eyes | <6 months | 22 | −15 ± 9 | P = 0.61 | −16 ± 10 | |
| | ≧6 months | 48 | −17 ± 10 | | | |

*Reported values for the rate of refractive growth are mean ± standard deviation.

For eyes with surgery at less than 6 months of age versus those with surgery at an older age, the relative difference of calculated rate of refractive growth was less for the RRG3 model (P=0.12 for pseudophakes, P=0.61 for aphakes) than for the RRG model (P<0.01 for pseudophakes, P=0.11 for aphakes) or for the RRG2 model (P=0.04 for pseudophakes, P=0.51 for aphakes) (Tables 3, 4).

TABLE 3
Comparison of mean RRG, RRG2, and RRG3 values in pseudophakes.

Mean Rate of Refractive
Growth (D)

| Model | Age <6 months | Age ≧6 months | P value |
|---|---|---|---|
| RRG | −3.3 ± 1 | −5.5 ± 3 | <0.01 |
| RRG2 | −4.9 ± 2 | −6.6 ± 3 | 0.04 |
| RRG3 | −11 ± 4 | −14 ± 7 | 0.12 |

*Reported values for rate of refractive growth are mean ± standard deviation. “Age” refers to the age at surgery.

TABLE 4
Comparison of mean RRG, RRG2, and RRG3 values in aphakes.

Mean Rate of Refractive
Growth (D)

| Model | Age <6 months | Age ≧6 months | P value |
|---|---|---|---|
| RRG | −4.9 ± 3 | −6.5 ± 4 | 0.11 |
| RRG2 | −6.6 ± 4 | −7.3 ± 5 | 0.51 |
| RRG3 | −15 ± 9 | −17 ± 10 | 0.61 |

*Reported values for rate of refractive growth are mean ± standard deviation. “Age” refers to the age at surgery.

Backward stepwise multiple regression provided an overall model P value of 0.001 and R² of 0.11. Log MAR BCVA (P=0.13), presence of an IOL (P=0.01), and calculated initial adjusted aphakic refraction (P=0.03) contributed to the model.

This study showed that the RRG3 values were not significantly different in infants less than 6 months of age versus 6 months of age or older at the time of surgery for either pseudophakic or aphakic eyes. This finding demonstrated that the optical artifact due to the vertex distance was a reason for the previously observed age-related difference in mean values for RRG and for RRG2.

It is also found that log MAR BCVA was negatively correlated with RRG3. As vision got worse, RRG3 became more negative. However, because BCVA is the long-term result of both good image quality on the retina and proper management of amblyopia, BCVA is not a direct substitute for the effect of image quality. In addition, because unilateral cataract patients have a much greater rate of amblyopia than those with bilateral cataracts, this apparent correlation between BCVA and RRG3 may be further confounded by laterality of the cataract.

No correlation between any of the following factors and RRG3 were found: age at surgery, sex, presence of glaucoma, and uni-versus bi-laterality of the surgery. Finding RRG3 to be independent of age at surgery is especially helpful because this one model can be used for all ages instead of needing to use separate models for patients of different ages.

It is also observed the mean RRG3 value was significantly less negative in pseudophakes than in aphakes. Previous studies in both children [29, 22] and monkeys[3, 21] found that pseudophakic eyes had less axial elongation than aphakic eyes. However, measurements of axial length have shown no significant difference over time between pseudophakic eyes and their fellow unoperated eyes[5]. This suggests that most pseudophakic eyes grow normally and are consequently expected to have a large myopic shift.

### EXAMPLE 2

### Prophetic Example to Validate the W Formula for Determining IOL Power

The Infant Aphakia Treatment Study (IATS) is a multi-center study with many children who have had cataract surgery and long-term follow-up. 114 infants who had unilateral congenital cataract surgery and were randomly assigned to either aphakia (no IOL implant) or pseudophakia (with an IOL implant) as subjects of the IATS. Their preoperative eye biometrics, IOL data, ages at measurements, and postoperative refractions were collected. The W formula will be used to predict the IOL powers for each child for each goal post-operative refraction, just as the other various IOL formulas were used pre-operatively to choose their actual IOL powers. The accuracy of the post-operative refraction using the W and RRG3 formula will be compared to the other currently accepted and used IOL formulas. The mean absolute error of predicted refraction (MAE) for all analyzed formulae, as calculated from preoperative biometry and IOL data, with a corrective factor for growth of the eye based on age at surgery and age at refraction measurement (calculated using RRG3).

### REFERENCE

1. Wirth M G, Russel-Eggitt I M, Craig J E, Elder J E, Mackey D A: Aetiology of congenital and pediatric cataract in an Australian population. Br J Ophthalmol 2002, 86:782-786.
2. Johan Z, Paul B M, Abdulaziz A, Saleh A M, David T W: Pediatric intraocular lens implantation: surgical results and complications in more than 300 patients. Ophthalmology 1998, 105:112-119.
3. Lambert S R, Lynn M, Drews-Botsch C: Intraocular lens implantation during infancy: perceptions of parents and the American Association for Pediatric Ophthalmology and Strabismus members. J AAPOS 2003, 7:400-405.
4. Wilson M E, Bartholomew L R, Trivedi R H: Pediatric cataract surgery and intraocular lens implantation: practice, styles and preferences of the 2001 ASCRS and AAPOS memberships. J Cataract and Refractive Surgery 2003, 29:1811-1820.
5. Kora Y, Suzuki Y, Inatomi M, Ozawa T, Fukado Y: A simple modified SRK formula for severely myopic eyes. Ophthalmic Surg 1990, 21:266-271.
6. Maya E T, Omer T, Steven M A, Monte A D M: Discrepancies between intraocular lens implant power prediction formulas in pediatric patients. Ophthalmology 2007, 114:383-386.
7. Maya E T, Steven M A, Monte A D M: Intraocular lens power calculation in children. Survey of Ophthalmology 2007, 52:474-482.
8. Sander D R, Retzlaff J, Kraff M C: Comparison of the SRK II formula and other second generation formulas. J Cataract and Refractive Surgery 1988, 14:36-41.
9. Sanders D R, Retzlaff J A, Kraff M C: Comparison of SRK/T formula and other theoretical and regression formula. J Cataract and Refractive Surgery 1990, 16:341-346.
10. Dang M S, Raj Sunder: SRK II formula in the calculation of intraocular lens power. Br J Ophthalmol 1989, 73:823-826.
11. Gordon R A, Donzis P B: Refractive development of the human eye. Arch Ophthalmol 1985, 103:785-789. Awner S, Buckley E G, DeVaro J M: Unilateral pseudophakia in children under 4 years. J Pediatric Ophthalmol Strabismus 1996, 33:230-236.
12. McClatchey S K, Parks M M: Theoretic refractive changes after lens implantation in childhood. Ophthalmology 1997, 104(11):1744-1751.
13. McClatchey S K, Parks M M: Myopic shift after cataract removal in childhood. J Pediatric Ophthalmol Strabismus 1997, 34:88-95.
14. Holladay J, Cravy T, Koch D: Calculating the surgically induced refractive change following ocular surgery. J Cataract and Refractive Surgery 1992, 18:429-443.
15. Olsen T, Dam-Johansen M: Refractive results after phacoemulsification and ECCE. A comparative study. Acta Ophthalmol 1993, 71:382-387.
16. Daniel E N, David A P, Sara M B, Richard L G: Accuracy of intraocular lens calculations in infants and children undergoing cataract surgery. J AAPOS 2005, 9:160-165.
17. Young C L, Hyun S K: Clinical Symptoms and visual outcome in patients with presumed congenital cataract. J Pediatric Ophthalmol Strabismus 2000, 37:219-224.
18. Haargaard B, Wohlfahrt J, Fledelius H C, Rosenberg T, Melbye M: A Nationwide Danish study of 1027 cases of congenital/infantile cataracts: etiological and clinical classifications. Ophthalmology 2004, 111:2292-2298.
19. Teresa C C, Lini S B, David S W: Complications of pediatric lensectomy in 193 eyes. Ophthalmic Surgery, Laser and Imaging 2005, 36:6-13.
20. Andreo L K, Wilson M E, Saunders R A: Predictive value of regression and theoretical IOL formulas in pediatric intraocular lens implantation. J AAPOS 1997, 34:240-243.
21. Lambert S R, Buckley E, Plager D: Unilateral intraocular lens implantation during the first six months of life. JAAPOS 1999, 3:344-349.
22. McClatchey S K, Dahan E, Maselli E: A comparison of the rate of refractive growth in paediatric aphakic and pseudophakic eyes. Ophthalmology 2000, 107:118-122.
23. Tromans C, Haigh P M, Biswas S, Lloyd I C: Accuracy of intraocular lens power calculation in pediatric cataract surgery. Br J Ophthalmol 2001, 85:939-941.
24. Superstein R, Archer S M, Del Monte M A. Minimal myopic shift in pseudophakic versus aphakic pediatric cataract patients. J AAPOS 2002; 6:271-6.
25. Astle W F, Ingram A D, Isaza G M, Echeverri P. Paediatric pseudophakia: Analysis of intraocular lens power and myopic shift. Clin Experiment Ophthalmol 2007; 35:244-51.
26. McClatchey S K. Refractive changes after lens implantation in childhood: Author's reply. Ophthalmology 1998; 105:1572-4.
27. McClatchey S K, Hofmeister E M. The optics of aphakic and pseudophakic eyes in childhood. Surv Ophthalmol 2010; 55:174-82.
28. Eibschitz-Tsimhoni M, Archer S M, Del Monte M A. Intraocular lens power calculation in children. Sury Ophthalmol 2007; 52:474-82.
29. Krishnamurthy R, Vanderveen D K. Infantile cataracts. Int Ophthalmol Clin 2008; 48:175-92.

We claim:

1. A method for predicting initial postoperative IOL power of a patient undergone IOL surgery, comprising:
a. measuring axial length of an eye of a patient;
b. measuring the cornea curvature of said eye;
c. choosing an IOL to be implanted; and
d. calculating the predicted initial postoperative power of the pseudophakic eye using the W system of formulas.

2. The method of claim 1, wherein said axial length is measured using an A-scan device or a B-scan device.

3. The method of claim 1, wherein said cornea curvature of said eye is measured using a Keratometer or a corneal topography device.

4. The method of claim 1, wherein step (d) further comprising: deriving a radius of curvature of the anterior cornea (Rak), a curvature of the posterior cornea (Rpk), and a thickness of the cornea (K_t) and an anterior chamber depth (ACD) as a function of the measured axial length.

5. The method of claim 4, wherein said the anterior cornea (Rak) is no greater than 8.9 mm.

6. A method to predicting refractive growth of a pediatric patient undergoing IOL surgery, comprising:
a. entering the age of the patient at the time of the surgery and a desired postoperative refraction;
b. selecting an age for the refractive growth prediction; and
c. calculating predicted refraction of the patient's eye at the selected age using
$$RRG 3 = \frac{{AdjAR}_{2} - {AdjAR}_{1}}{log ( {AdjAge}_{2} ) - log ( {AdjAge}_{1} )}$$
Wherein RRG3 is the rate of refractive growth, AdjAR₁ is the desired postoperative refraction, AdjAR₂ is adjusted aphakic refraction at selected age AdjAge₁ is the patient's age at the time of surgery plus 0.6 years, AdjAge₂ is the selected age plus 0.6 years.

7. The method of claim 6, wherein said adjusted aphakic refraction is IOL power for emmetropia at the natural lens plane.

8. The method of claim 6, wherein RRG3 is the difference in the adjusted aphakic refractions divided by the difference in the logarithms of the adjusted ages.

9. The method of claim 8, wherein said RRG3 is −13±6.
